(19) United States
(12) Patent Application Publication
(10) Pub. No.: US 2003/0091619 A1
(43) Pub. Date: May 15, 2003

(54) Temporomandibular joint patch

(76) Inventor: Jamison R. Spencer, Boise, ID (US)

Correspondence Address: ROBERT L. SHA VER, DYKAS & SHA VER, LLP, P.O. BOX 877, BOISE, ID, 83701-0877, US

(21) Appl. No.: 09/992,723

(22) Filed: Nov. 14, 2001

(51) Int. Cl. A61K 9/70

(52) U.S. Cl. 424/449

(57) ABSTRACT

A system and method for decreasing pain in dental patients by use of a transdermal patch placed over the temporomandibular joint. To provide relief from jaw pain and/or to prepare a patient for an upcoming dental procedure, a patch containing a pre-selected medication is placed over the temporomandibular joint in front of a patient's ear. The medication from the patch passes through the skin and into the jaw lessening the pain and soreness connected with traditional dental procedures from having one's mouth opened for an extended period of time.

### BACKGROUND OF THE INVENTION

1. Field of the Invention.

The present invention generally relates to a method of and product for providing local pain relief to patients suffering from jaw pain or undergoing dental procedures, and more specifically to treatments for pain in the temporomandibular joint.

2. Background Information.

One of the chief complaints of patients during and after visiting a dental office is of pain and stiffness from holding one's mouth open for so long. Although local anesthetics are injected or otherwise administered to numb the teeth and gingival tissue of the patient, this is not effective in providing relief to the muscles that open and close the mouth or are connected to the jaw joints. Furthermore, patients may also have an onset or sudden flash of pain after they have left the dental office. Although a dentist could prescribe an oral medication, these types of medication generally are slower in effect and may be ineffective or less effective depending upon the size of the person to whom the medication is delivered. Also, an analgesic of sufficient strength to provide this type of pain control would tend to be very sedative restricting the patient's ability to drive a car or perform other activities requiring attentiveness.

Topical applications of medications both in cream and in gel form have been used for years. Over-the-counter products include cortisone, aspirin and others. The problem with cream and/or gel applications is that patients are left on their own to administer the products. Often errors occur in the amount, location and manner of the medication applied and as a result, the appropriate therapeutic properties are not achieved.

The use of creams and/or gels in the professional dental setting is inappropriate due to concerns of cross-contamination between patients. Having a tube of gel or cream that is opened, used on a patient, closed, then reopened and used on another patient is potentially unsanitary and therefore unacceptable. However, such creams are effective in providing relief to patients. Therefore, what is needed is a method and/or device for delivering a measured amount of medication capable of providing safe, sanitary, and effective relief to patients who would otherwise suffer pain and discomfort. Another object of the invention is to provide a dosage of medication tailored to be used on a patient's temporomandiblar joint.

### SUMMARY OF THE INVENTION

This invention functions to provide relief to and prevent pain in dental patients by placing a patch containing a desired amount of medication over the temporomandibular joint area, in front of a patient's ear. The medication is carried from the patch through the patient's skin to the desired area to be affected. This invention results in greater patient comfort during dental procedures and reduces pain and discomfort following dental procedures. This patch has several embodiments that can be utilized. However, the two principal ways will be discussed.

In a first embodiment, a high dose concentration of the medication is placed on the patch. Ideally this would be done outside of the dentist's office, and individually prepackaged for use. While at the dentist's office, the dental assistant will simply select the patch having the desired quantity and potency of medication, open the package and place the patch on the desired location of the patient's body. This patch will be most effective in treating and alleviating dental pain when placed over the area of the temporomandibular joint, in front of the patient's ear. After the patch is placed over the area of the temporomandibular joint, the medication penetrates the skin reaching into the muscles, including the masseter and the lateral pterygoid muscles, and the desired area to be affected. This provides relief from pain and discomfort while the dentist performs the procedure for which the patient has come to the office.

In the second embodiment, a patch with a lower dosage of medication can be used either in the office and/or sent home with the patient. When the patient is at home and experiences a relapse of pain, the patient can simply place the patch on the same area of the temporomandibular joint, immediately in front of their ear, whereby they can receive relief from the pain of the joint or related muscles and discomfort they are experiencing.

These embodiments are superior to the present embodiments of pain relief in that there is no needle involved, therefore reducing both the dangers to the patient and to the administrator of the medication of possible needle sticks, nerve damage, and hematoma formation. Furthermore, there is no mess or cross contamination issue because the patient is never contacted with an open tube of gel and/or cream. There is no problem of over or under dosing the amount of medication because the selected dosage of medication is enclosed within the sealed patch. This embodiment does away with the problems associated with tube and gel medications; the patch device is more sanitary; the dosage is controlled; and the device is simple to use.

### BRIEF DESCRIPTION OF THE DRAWINGS

FIG. 1 is a cross-sectional view of the TMJ patch.

FIG. 2 is a bottom planar view of the TMJ patch.

FIG. 3 is a profile view of a human figure showing the temporomandibular joint and location of placement of the TMJ patch.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed. On the contrary, the invention is to cover all modifications, alternative constructions, and equivalents falling within the spirit and scope of the invention as defined in the claims.

The temporomandibular joint (TMJ) is located at the junction of the temporal portion of the human cranium and the mandible or jawbone. This joint is one of most frequently used joints in the human body involved in talking, chewing, swallowing, eating, and breathing. The TMJ is a ganglyarthroidal joint. Its movements are controlled by muscles.

People feel pain in the temporomandibular joint when these muscles are overexerted or when the joint is misaligned or malfunctioning in a way that places excess pressure on the joint or muscles, such as a spasm.

One of the common times when pain and soreness is felt is when the muscles that surround the TMJ are stretched or contracted for a relatively long period of time, such as when a person undergoes a dental procedure. As little as five to ten minutes of either stretching or contracting the muscles without a break can cause pain and soreness. Having the mouth open for a long period of time, especially when one is undergoing a lengthy dental procedure, stretches certain muscles and contracts opposing muscles in such a fashion that pain and soreness result. Having the mouth open in such a way is not a normal activity and thus these tissues are quickly fatigued.

Another common type of pain associated with the TMJ occurs in persons who have TMJ dysfunction or disorder. This occurs when the TMJ is misaligned or malfunctioning in such a way that the pressure on the joint and surrounding tissues is increased. When this occurs a patient may feel pain in their ear, cheek, jaw, teeth, or even have a headache behind their eyes or in other areas of the head, face and neck. Often times people will have short-term acute flare-ups of such pain or chronic pain or dysfunction.

It is an object of this invention to provide a means for providing safe, sanitary and effective relief to such persons, both in the dental office and at home. One preferred embodiment is shown in the figures.

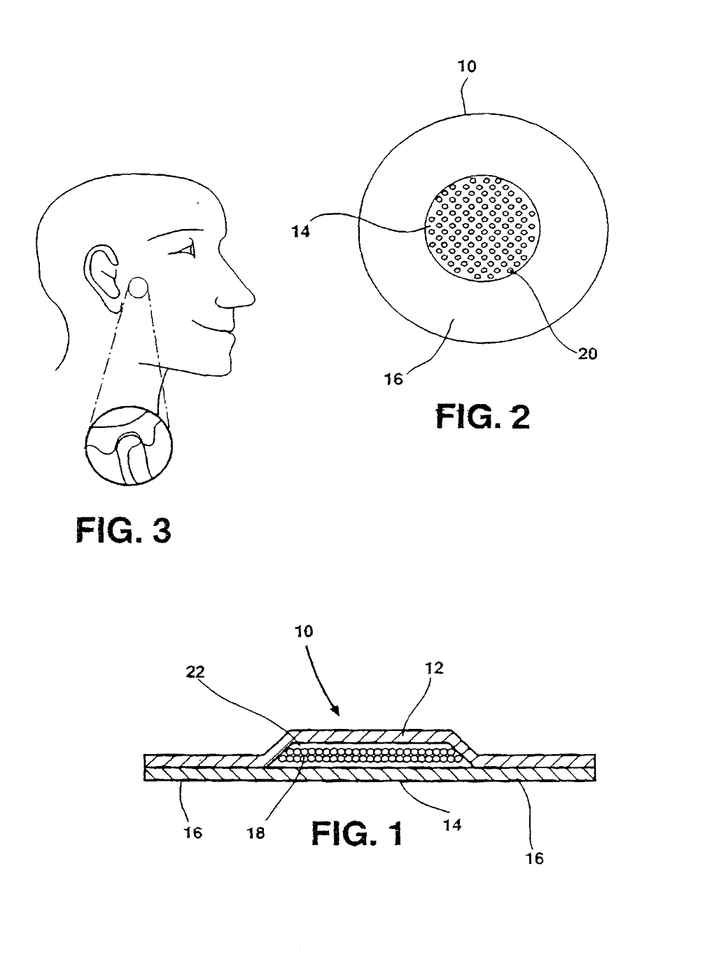

In FIG. 1 a patch is shown which includes a dermal contact layer 14 and a backing layer 12. The dermal contact layer is permeable to a medication 22 and is attached to the backing layer 12 by conventional means. The patch having a backing layer 12 and a dermal contact layer 14 is prepared by the placement of a desired amount and type of medication 22 between the two surfaces. The medication to be used could be an analgesic, such as Benzocaine, or Lidocaine, an anti-inflammatory, such as Ibuprofen or Ketoprofen, a vasoconstricting medication such as epinephrine, or any other desired medication, including a so-called natural or herbal supplement such as chondroitin sulfate. In some situations, the use of a delivery-enhancing device such as PLO gel (pluronic lecithin organogel) would be appropriate for facilitating the passage of the medication though the patient's skin.

In some applications, the medication would be contained within an absorbent layer 18 made of any conventional pharmacologically absorbent material, which allows for release of a medication through the permeable dermal contact layer 14

The periphery of the dermal contact layer 14 contains an adhesive 16 which allows for the patch to be removeably attached to the dermis of a patient, and keeps the patch on the patient's skin, particularly in the area of the temporomandibular joint.

FIG. 2 demonstrates a bottom view of a patch wherein the dermal contact layer of the patch 14 is made permeable to the passage of a medication by means of a plurality of pores 20 located within the dermal contact layer. The periphery of the dermal contact layer 14 contains an adhesive 16 for dermal attachment to a patient.

In the preferred embodiment patches would be commercially prepared in advance and packaged for individual use either in the dentist's office or by the patient at home. Various dosages would be made available, with the desired combination of analgesic, and/or anti-inflammatory and dermal penetrant. These patches would be useful for treating, both acute and chronic types of TMJ pain and could be obtained by prescription through the medical or dental office or over the counter.

In the dental office situation, the dentist or an employee working under the supervision of the dental professional, would place the desired amount of medication onto the patient by simply selecting the patch to be used, opening the package, and placing the patch in front of the patient's ear over the area of the temporomandibular joint. After putting the patch into place, the medication would penetrate the skin and enter the desired area. After a period of time, during which the medication has passed through the patient's skin to the desired area, the desired area of the patient's body would be anesthetized and relaxed to the point where the dental professional could then perform the procedure with significantly less discomfort for the patient. A preferred combination of ingredients for the TMJ patch comprises: an analgesic such as Lidocaine combined with an anti-inflammatory medication such as Ibuprofin. A range of useful ingredients for the TMJ patch comprises: local anesthetics, analgesics, anti-inflammatories, in prescription or over-the-counter strengths, and various herbal treatments as may be appropriate.

This patch could be used in office dental visits, including fillings, crown and bridge procedures, extractions, periodontal procedures, orthodontic, endodontic, or any other dental or oral surgery procedure that would require the patient to hold their mouth open for more than a few minutes. A patch containing a medicinal mixture of 20% Benzocaine, 6% Lidocaine, and 4% Tetracaine in PLO gel is an excellent combination for use in this environment.

In another application, a lower dose package could be given to the patient when they leave the dental office following the procedure. The patient could then take these patches to their home where they could use these medicated patches when pain returned or when the inflammation and myalgia caused them discomfort. The patient would simply need to place the pre-medicated patch in the appropriate area and wait for the medication to permeate to the desired area.

While there is shown and described the present preferred embodiment of the invention, it is to be distinctly understood that this invention is not limited thereto but may be variously embodied to practice within the scope of the following claims.

I claim:

1. A system for delivering medication to a desired location transdermally comprising:
a patch comprising a backing layer, said backing layer having a backing layer periphery, and a dermal contact layer, with said dermal contact layer being configured to be permeable to a medication, said dermal contact layer further comprising a periphery, said backing layer being attached to said dermal contact layer at said backing layer periphery, with said dermal contact layer including an adhesive surface at the periphery of said dermal contact layer for dermal attachment to a patient above said desired location; and
at least one medication sealed between said backing layer and said dermal contact layer;
wherein said patch is configured for dermal attachment to said patient and said medication is configured to pass through said dermal contact layer into said desired location.

2. The system of delivering medication of claim 1 wherein said medication further comprises a delivery enhancing component for aiding the transdermal passage of said medication from said dermal contact layer to said desired location.

3. The system of delivering medication of claim 1 wherein said adhesive surface is a pressure sensitive adhesive whereby said patch may be dermally affixed.

4. The system of delivering medication of claim 1 wherein said medication is one or more pharmacological agent selected from the group comprising anesthetics, analgesics, vasoconstrictors, anti-inflammatory medications.

5. The system of delivering medication of claim 1 wherein said medication is a natural supplement such as glucosamine chondroitan sulfate.

6. The system of delivering medication of claim 1 wherein said medication is one or more pharmacological agent selected from the group comprising anesthetics, analgesics, vasoconstrictors, anti-inflammatory medications, and a natural supplement such as glucosamine chondroitan sulfate.

7. The system of delivering medication of claim 2 wherein said delivery enhancing component is the transdermal pharmacological agent pluronic lecithin organogel (PLO gel).

8. The system of delivering medication of claim 1 wherein said desired location is the area of the temporomandibular joint.

9. The system of delivering medications of claim 1, which includes an absorptive material between, said dermal contact layer and said backing layer.

10. The system of delivering medication of claim 1 in which said dermal contact layer further comprises a plurality of medication pores, for allowing passage of said medication from between said backing layer and said dermal contact layer, to said temporomandibular joint area.

11. A medication system for transdermally delivering medication to a temporomandibular joint comprising:
a patch comprising a backing layer, said backing layer having a backing layer periphery, and a dermal contact layer, with said dermal contact layer being configured to be permeable to a medication, said dermal contact layer further comprising a periphery, said backing layer being attached to said dermal contact layer at said backing layer periphery, with said dermal contact layer including an adhesive surface at the periphery of said dermal contact layer for dermal attachment to a patient above said temporomandibular joint; and
at least one medication sealed between said backing layer and said dermal contact layer;
wherein said patch is configured for dermal attachment to said patient at said temporomandibular joint and said medication is configured to pass through said dermal contact layer into muscles surrounding said temporomandibular joint.

12. A medication system for delivering medication to a temporomandibular joint area transdermally comprising:
a patch comprising a backing layer with a backing layer periphery, and a dermal contact layer, said dermal contact layer configured to be permeable to a medication, said dermal contact layer further comprising a dermal contact layer periphery, said backing layer being attached to said dermal contact layer at said backing layer periphery, said dermal contact layer periphery further comprising an adhesive surface, for dermal attachment to a patient above the temporomandibular joint area; and
at least one medication sealed between said backing layer and said dermal contact layer, said medication further comprising one or more pharmacological agents selected from the group comprising anesthetics, analgesics, vasoconstrictors, anti-inflammatory medications and herbal medications;
wherein said patch is configured for dermal attachment to said patient and said medication is configured to pass through said dermal contact layer into said temporomandibular joint area.

13 The medication delivery system of claim 9 wherein said medication is of a high potency whereby full local anesthetic relief is achieved.

14. The medication delivery system of claim 9 wherein said medication is of a low potency whereby relief from pain and discomfort is achieved.

15. A method of administering medications to a jaw having a temporomandibular joint area comprising:
preparing a patch for applying a medication; and
applying said patch on a dermal surface adjacent to the temporomandibular joint area;
whereby said medication passes transdermally from said patch into an area surrounding said temporomandibular joint.

16. A method of treating jaw pain comprising:
preparing a patch for applying a medication, wherein said patch further comprises a backing layer having a backing layer periphery, and a dermal contact layer, with said dermal contact layer being configured to be permeable to a medication, said dermal contact layer further comprising a periphery, said backing layer being attached to said dermal contact layer at said backing layer periphery, with said dermal contact layer including an adhesive surface at the periphery of said dermal contact layer for dermal attachment to a patient; with at least one medication of a desired type and amount, said medication sealed between said backing layer and said dermal contact layer; and
applying said patch on a dermal surface adjacent to a temporomandibular joint;
whereby said medication passes transdermally from said patch into an area surrounding said temporomandibular joint, thereby relieving jaw pain and soreness.

17. The method of treating jaw pain in humans of claim 14, which further includes adding a delivery enhancement component to, said patch.

18. The method of treating jaw pain in humans of claim 14 wherein said patch further comprises a high potency medication whereby full local anesthetic relief is achieved.

19. The method of treating jaw pain in humans of claim 14 wherein said patch further comprises a medication of a dosage and type sufficient to relieve pain and discomfort.

20. A method of treating and alleviating jaw pain in humans comprising:
preparing a patch for applying medication wherein said patch for applying medication comprises a backing layer having a backing layer periphery, and a dermal contact layer, with said dermal contact layer being configured to be permeable to a medication, said dermal contact layer further comprising a periphery, said backing layer being attached to said dermal contact layer at said backing layer periphery, with said dermal contact layer including an adhesive surface at the periphery of said dermal contact layer for dermal attachment to a patient; with a medication comprising at least one or more pharmacological agent selected from the group comprising anesthetics, analgesics, vasoconstrictors, anti-inflammatory medications, and herbal medications wherein such medication is of such a potency whereby relief from pain and discomfort is achieved, said medication sealed between said backing layer and said dermal contact layer; and
applying said patch for applying medication on a dermal surface adjacent to a temporomandibular joint;
whereby said medication passes transdermally from said patch into an area surrounding said temporomandibular joint, thereby relieving jaw pain and soreness.

21. A method of delivering anesthesia to dental patients comprising:
preparing a patch applying a medication, said patch comprises a backing layer having a backing layer periphery, and a dermal contact layer, with said dermal contact layer being configured to be permeable to a medication, said dermal contact layer further comprising a periphery, said backing layer being attached to said dermal contact layer at said backing layer periphery, with said dermal contact layer including an adhesive surface at the periphery of said dermal contact layer for dermal attachment to a patient, with a medication comprising at least one or more pharmacological agent selected from the group comprising anesthetics, analgesics, vasoconstrictors, and anti-inflammatory medications sealed between said backing layer and said dermal contact layer; said pharmacological agent of a sufficient potency whereby desired local relief is achieved; and
applying said patch on a dermal surface adjacent to a temporomandibular joint;
whereby said medication passes transdermally from said patch into an area surrounding said temporomandibular joint, providing desired theraputic effects upon said area.

22. The method of treating jaw pain in humans of claim 18 which further includes preparing said patch with a delivery enhancing component.
